(19) United States
(12) Patent Application Publication
(10) Pub. No.: US 2025/0114125 A1
(43) Pub. Date: Apr. 10, 2025

(54) SLIM AND ERGONOMIC HAND-HELD ENDOSCOPIC KIT WITH AN INJECTION SHEATH

(71) Applicant: MicronVision Corp., Bellevue, WA (US)

(72) Inventor: Xiaolong OUYANG, Bellevue, WA (US)

(21) Appl. No.: 18/982,803

(22) Filed: Dec. 16, 2024

Related U.S. Application Data
(63) Continuation-in-part of application No. 18/374,740, filed on Sep. 29, 2023.
(63) Continuation-in-part of application No. 18/039,209, filed on May 26, 2023, now Pat. No. 12,181,460.
(63) Continuation-in-part of application No. 18/626,133, filed on Apr. 3, 2024.
(60) Provisional application No. 63/613,772, filed on Dec. 22, 2023.
(60) Provisional application No. 63/620,838, filed on Jan. 14, 2024.
(60) Provisional application No. 63/623,236, filed on Jan. 20, 2024.
(60) Provisional application No. 63/624,086, filed on Jan. 23, 2024.
(60) Provisional application No. 63/554,976, filed on Feb. 17, 2024.
(60) Provisional application No. 63/555,552, filed on Feb. 20, 2024.
(60) Provisional application No. 63/556,151, filed on Feb. 21, 2024.
(60) Provisional application No. 63/556,712, filed on Feb. 22, 2024.
(60) Provisional application No. 63/575,761, filed on Apr. 7, 2024.
(60) Provisional application No. 63/634,487, filed on Apr. 16, 2024.
(60) Provisional application No. 63/639,679, filed on Apr. 28, 2024.
(60) Provisional application No. 63/640,000, filed on Apr. 29, 2024.
(60) Provisional application No. 63/640,113, filed on Apr. 29, 2024.
(60) Provisional application No. 63/659,483, filed on Jun. 13, 2024.
(60) Provisional application No. 63/659,455, filed on Jun. 13, 2024.
(60) Provisional application No. 63/660,531, filed on Jun. 16, 2024.
(60) Provisional application No. 63/663,285, filed on Jun. 24, 2024.
(60) Provisional application No. 63/663,339, filed on Jun. 24, 2024.
(60) Provisional application No. 63/668,286, filed on Jul. 7, 2024.
(60) Provisional application No. 63/672,949, filed on Jul. 18, 2024.
(60) Provisional application No. 63/683,198, filed on Aug. 14, 2024.
(60) Provisional application No. 63/685,682, filed on Aug. 21, 2024.
(60) Provisional application No. 63/693,589, filed on Sep. 11, 2024.
(60) Provisional application No. 63/713,926, filed on Oct. 30, 2024.
(60) Provisional application No. 63/718,614, filed on Nov. 9, 2024.
(60) Provisional application No. 63/608,316, filed on Dec. 11, 2023.
(60) Provisional application No. 63/674,055, filed on Jul. 22, 2024.

(51) Int. Cl. A61B 17/34

(52) U.S. Cl. CPC A61B 17/3478; A61B 17/3496

(57) ABSTRACT

A slim and ergonomic kit for injecting an agent into a patient's internal passageway, for example to treat conditions such as stress-induced incontinence (SUI) includes a sheath and a cannula with distal portions dimensioned to form a tubular space for fluid flow between the outside of the cannula and the inside of the sheath. Preferably, the sheath is molded as a single unit comprising a sheath tube, a needle channel, and a needle entry port. A display can be mechanically mounted to a handle to which the cannula is secured, or the handle can wirelessly transmit images taken with an imaging module at the distal end of the cannula to a remote display.

### REFERENCE TO RELATED APPLICATIONS

This application is a continuation in-part of and incorporates by reference each of parent U.S. applications (i) Ser. No. 18/374,740 filed Sep. 29, 2023, (ii) Ser. No. 18/039,209 filed Dec. 16, 2022, and (iii) Ser. No. 18/626,133 filed Apr. 5, 2024.

This application claims the benefit of and incorporate by reference each of the above-referenced parent applications and the following U.S. provisional patent applications:
Ser. No. 63/613,772, filed Dec. 22, 2023
Ser. No. 63/620,838, filed Jan. 14, 2024
Ser. No. 63/623,236, filed Jan. 24, 2024
Ser. No. 63/624,086, filed Jan. 25, 2024
Ser. No. 63/554,976, filed Feb. 17, 2024
Ser. No. 63/555,552, filed Feb. 20, 2024
Ser. No. 63/556,151, filed Feb. 21, 2024
Ser. No. 63/556,712, filed Feb. 22, 2024
Ser. No. 63/575,761, filed Apr. 8, 2024
Ser. No. 63/634,487, filed Apr. 16, 2024
Ser. No. 63/639,679, filed Apr. 28, 2024
Ser. No. 63/640,000, filed Apr. 29, 2024
Ser. No. 63/640,113, filed Apr. 29, 2024
Ser. No. 63/659,483, filed Jun. 13, 2024
Ser. No. 63/659,455, filed Jun. 13, 2024
Ser. No. 63/660,531, filed Jun. 16, 2024
Ser. No. 63/663,285, filed Jun. 24, 2024
Ser. No. 63/663,339, filed Jun. 24, 2024
Ser. No. 63/668,286, filed Jul. 7, 2024
Ser. No. 63/672,949, filed Jul. 18, 2024
Ser. No. 63/683,198, filed Aug. 14, 2024
Ser. No. 63/685,682, filed Aug. 21, 2024
Ser. No. 63/693,589, filed Sep. 11, 2024
Ser. No. 63/713,926, filed Oct. 30, 2024
Ser. No. 63/718,614, filed Nov. 9, 2024
Ser. No. 63/608,316, Dec. 27, 2023
Ser. No. 63/674,055, filed Jul. 22, 2024
Ser. No. 63/734,073, filed Dec. 14, 2024

### FIELD

This patent specification relates to portable, hand-held endoscopes and access sheaths.

### BACKGROUND

Portable, hand-held endoscopes are widely used in medical procedures and are gradually superseding conventional endoscopes that are reusable and are considerably more expensive and require thorough decontamination between patients, which in turn requires specialist personnel and facilities.

Hand-held endoscopes are referred to in the following U.S. patents and U.S. Published patent applications, each of which is hereby incorporated by reference: US20230200634-A1, US20230128303-A1, US20230117151-A1, US20220296090-A1, US20220273165-A1, US20220211252-A1, US20220142460-A1, US 20220079418-A1, US20210401277-A1, US20210338052-A1, US20210307591-A1, US20210251789-A1, US20200221932-A1, US20200077880-A1, US20190282071-A1, US20190216325-A1, US20180256009-A1, US20180132700-A1, US 20170215699-A1, US20170188793-A1, US20170188795-A1, US20160367119-A1, US20160174819-A1, US20150164313-A1, US20150157387-A1, US20150150441-A1, US20120289858-A1, US20120100729-A1, US20110270179-A1, US 20110009694-A1, US20100284580-A1, US20100286477-A1, US20100121139-A1, US20100121142-A1, US20100121155-A1, US20110009694-A1, US20110006364-A1, U.S. Pat. Nos. 11,684,248-B2, 11,395,579-B2, US20220168035-A1 U.S. Pat. Nos. 11,330,973-B2, 11,071,442-B2, 11,013,396-B2, US20210137352-A1, US20210093169-A1, U.S. Pat. Nos. 10,874,287-B2, 10,869,592-B2, 10,524,636-B2, 10,441,134-B2 U.S. Pat. No. 10,426,320-B2, US20190261836-A1, U.S. Pat. Nos. 10,362,926-B2, 10,292,571-B2, 10,278,563-B2, US20190117050-A1, US20180271581-A1, US20180256009-A1, U.S. Pat. No. 10,045,686-B2, US20180206707-A9, US20180132701-A1, U.S. Pat. No. 9,895,048-B2, US20170188794-A1, US20170188795-A1, U.S. Pat. No. 9,649,014-B2, 9,622,646-B2, 9,468,367-B2, US20140288460-A1, US20140276207-A1, US20130296648-A1, U.S. Pat. No. 8,460,182-B2. An example of a sheath used with an optical rather than an electronically imaging endoscope is proposed in U.S. Pat. No. 7,758,497.

### SUMMARY

According to some embodiments, a kit is useful for medical procedures that includes injecting an agent at one or more injection sites in a patient's internal passageway. The kit comprises an injection sheath that includes (i) a sheath tube with open distal and proximal ends and a central axis, (ii) an injection needle channel with a central axis parallel to but radially spaced from the tube's central axis, and (iii) a needle entry port that is at a proximal portion of the sheath and is coupled to the needle channel; wherein the sheath tube, the needle channel, and the needle entry port are integrally molded as a single unit of a biocompatible plastic material. The kit's sheath further comprises a tubular cap that is fitted to the distal end of the sheath tube and has (i) a plurality of orientation landmarks that extend radially inwardly and are circumferentially and radially spaced from each other and (ii) a stopper that is axially proximal to the orientation landmarks. The kit further comprises an endoscope unit that includes (i) a tubular cannula with an internal channel open at a distal end of the cannula, (ii) an imaging module at the cannula's distal end, (iii) fluid inflow and fluid outflow ports at a proximal portion of the sheath, with the inflow port coupled to the cannula's internal channel; wherein the sheath tube rotatably fits over a distal portion of the cannula; wherein (i) the cannula is proximal to the stopper, (ii) the imaging module is proximal to and in a position to image the orientation landmarks and a field distal from the sheath, (iii) the proximal portion of the cannula, with the inflow and outflow ports, is proximal to the sheath, and (iv) the cannula and the sheath tube are dimensioned to provide a tubular space for fluid flow therebetween that is coupled to the outflow port and has an open distal end.

According to some embodiments, the kit can further include one of more of: (a) a handle to which the proximal portion of the cannula is releasably coupled mechanically and electrically, the handle comprising a hollow pistol grip extending in a downward direction and shaped and dimensioned to be grasped by a user's hand and having a removable end cap closing off a bottom portion of the grip; (b) a display mounted to the handle; (c) processor electronics housed in their entirety in the hollow grip and electrically coupled with the imaging module and the display to receive image data from the imaging module and to display images based thereon on the display; (d) the handle end cap can be removable without tools to enable the processing electronics to slide up into the handle grip and the cap is configured to re-attach to the grip so the grip encloses the entirety of the processing electronics; (e) the sheath can comprise a tube made of a medical grade metal that lines the needle channel to facilitate distal motion therein of an injection needle with a sharp distal end, wherein the metal tube is molded into the needle channel while molding the sheath tube, the needle port, and the sheath tube in the same mold; (f) the cannula's proximal portion can have a plurality of axially elongated stiffening ribs that extend radially relative to the central axis and are circumferentially spaced from each other; (g) the sheath's proximal portion can have a plurality of axially elongated stiffening ribs that extend radially relative to the central axis and are circumferentially spaced from each other; (h) the external diameter of the cannula's distal portion and the internal diameter of the sheath can differ by 0.5 mm to thereby form the tubular space for fluid flow therein; and (i) the sheath can have a diameter that is 6 mm when intersecting the needle channel and 5.4 mm elsewhere.

According to some embodiments, a method comprises: introducing into a patient's passageway an injection sheath with open distal and proximal ends and an injection needle channel with a central axis parallel to but radially spaced from a central axis of the sheath and a needle entry port at a proximal portion of the sheath coupled to the needle channel; including in a distal portion of the sheath a tubular cap that has a plurality of orientation landmarks extending radially inwardly and circumferentially and radially spaced from each other and a stopper that is axially proximal to the orientation landmarks; providing an endoscope inserted in the sheath and having an internal channel open at a distal end, an imaging module at the endoscope's distal end, and fluid inflow and fluid outflow ports at a proximal portion of the endoscope, with the inflow port coupled to the endoscope's internal channel; selectively rotating by hand the sheath and the endoscope relative to each other while the endoscope is proximal to the stopper, the imaging module is proximal to and in a position to image the orientation landmarks and a field distal from the sheath, the proximal portion of the endoscope, with the inflow and outflow ports, is proximal to the sheath; wherein the endoscope and the sheath are dimensioned to provide a tubular space for fluid flow therebetween coupled to the outflow port and having an open distal end; and passing an injection needle through the needle channel such that a sharpened distal end of the needle protrudes distally from the sheath and injecting an agent into tissue through the needle.

According to some embodiments, the method can further include one or more of: (a) lining the needle channel with a tube made of surgical grade metal to facilitate distal motion therein of the sharpened distal end of the needle; (b) viewing images based on image data from the image module on a display mounted to a handle to which the endoscope is releasably coupled; and (c) inserting processing electronics into the handle after removing without tools a bottom cap of the handle, closing the bottom cap to enclose the entirety of the processing electronics in the handle, carrying out a medical procedure with the kit, opening the bottom cap and removing the processing electronics from the kit, removing the display without tools, and disposing of the remainder of the kit as medical waste.

According to some embodiments, a kit is provided for a medical procedure that includes injecting an agent at one or more injection sites in a patient's internal passageway. The kit comprises: an injection sheath that comprises a sheath tune with open distal and proximal ends and a central axis, an injection needle channel with a central axis parallel to but radially spaced from the sheath's central axis, and a needle entry port that is at a proximal portion of the sheath and is coupled to the needle channel; wherein the sheath tube, the needle channel, and the needle entry port are integrally molded as a single unit of a biocompatible plastic material; wherein the sheath includes a tubular cap that is fitted to the distal end of the sheath tube and has a plurality of orientation landmarks that extend radially inwardly and are circumferentially and radially spaced from each other and a stopper that is axially proximal to the orientation landmarks; an endoscope unit that includes a tubular cannula with an internal channel open at a distal end of the cannula and an imaging module at the cannula's distal end; wherein the sheath and the cannula are configured to rotate by had relative to each other about the cannula's central axis and the sheath tube has an inside diameter greater than the cannula's outside diameter to thereby form a tubular space therebetween for passage of fluid; and a handle with an inflow port at a proximal end of the handle coupled to the cannula's internal channel and an outflow port extending above the handle and coupled to said tubular space; wherein the cannula is proximal to the stopper and the imaging module is proximal to and in a position to image the orientation landmarks and a field distal from the sheath.

According to some embodiments, the kit described in the immediately preceding paragraph can further include one or more of: (a) the handle can comprise a hollow pistol grip extending in a downward direction and shaped and dimensioned to be grasped by a user's hand and having a removable end cap closing off a bottom portion of the grip, and processor electronics housed in their entirety in the hollow grip and electrically coupled with the imaging module and the display to receive image data from the imaging module and to display images based thereon on the display; (b) the sheath further comprises a tube made of a medical grade metal that lines the needle channel to facilitate distal motion therein of an injection needle with a sharp distal end, wherein the metal tube is molded into the needle channel while molding the sheath tube, the needle port, and the sheath tube in the same mold; (c) the cannula's proximal portion has a plurality of axially elongated stiffening ribs that extend radially relative to the central axis and are circumferentially spaced from each other; (d) the sheath's proximal portion has a plurality of axially elongated stiffening ribs that extend radially relative to the central axis and are circumferentially spaced from each other; and (c) the external diameter of the cannula's distal portion and the internal diameter of the sheath differ by 0.5 mm to thereby form the tubular space for fluid flow therein.

The Summary pertains to the subject matter of the initially presented claims, which may change during prosecution of this patent application.

### BRIEF DESCRIPTION OF THE DRAWINGS

To further clarify the above and other advantages and features of the subject matter of this patent specification, specific examples of embodiments thereof are illustrated in the appended drawings. It should be appreciated that these drawings depict only illustrative embodiments and are therefore not to be considered limiting of the scope of this patent specification or the appended claims. The subject matter hereof will be described and explained with additional specificity and detail through the accompanying drawings in which:

FIG. 1 is a perspective view illustrating a slim and ergonomic hand-held kit of an endoscope and an injection sheath, according to some embodiments.

FIG. 2 is a side view of the kit or system of FIG. 1.

FIG. 3 is a side view and FIG. 4 is a perspective view of a cannula and an injection sheath of the system of FIG. 1.

FIG. 5 is an axial sectional view of a cannula and sheath, according to some embodiments.

FIG. 6 is an exploded perspective view of a cannula and sheath, according to some embodiments.

FIG. 7 is a partial side view of a cannula and sheath and partial axial section of a distal portion of the sheath, according to some embodiments.

FIG. 7A is an enlarged axial sectional view of a distal portion of a sheath, according to some embodiments.

FIGS. 7B-D are cross-sectional views of a cannula and sheath, according to some embodiments.

FIG. 8 is a view in the proximal direction of a distal end of the sheath and cannula, according to some embodiments.

FIG. 9 is a perspective view of a kit that includes a cannula and a sheath but no display mechanically mounted to the sheath, according to some embodiments.

FIG. 10 is a perspective exploded view of components of the kit of FIG. 9, according to some embodiments.

### DETAILED DESCRIPTION

A detailed description of examples of preferred embodiments is provided below. While several embodiments are described, the new subject matter described in this patent specification is not limited to any one embodiment or combination of embodiments described herein, but instead encompasses numerous alternatives, modifications, and equivalents. In addition, while numerous specific details are set forth in the following description to provide a thorough understanding, some embodiments can be practiced without some or all these details. Moreover, for the purpose of clarity, certain technical material that is known in the related art has not been described in detail to avoid unnecessarily obscuring the new subject matter described herein.

Individual features, components, and elements of one or several of the specific embodiments described herein can be used in or in combination with other described embodiments or with other features, components, and elements. Like reference numbers and designations in the various drawings indicate like elements.

A system described below is simple and ergonomic kit or system of a hand-held endoscope and an injection sheath that is particularly useful in medical procedures for treatment of stress-induced incontinence (SUI) but can be used in other procedures as well. The system provides orientation landmarks within a target cavity in a patient's body for visualizing the cavity and the orientation of the sheath, a gap between an imaging module and the distal end of the endoscope to keep a camera lens from touching target tissue that may obscure images, and separate channels for inflow into the patient's cavity and for outflow for aspiration of the cavity.

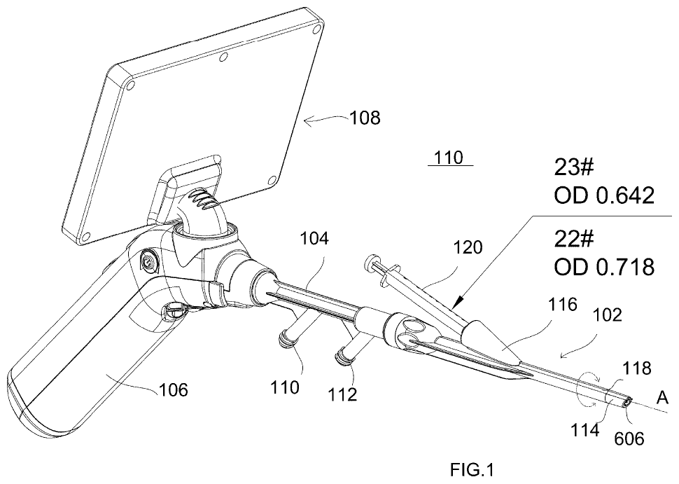

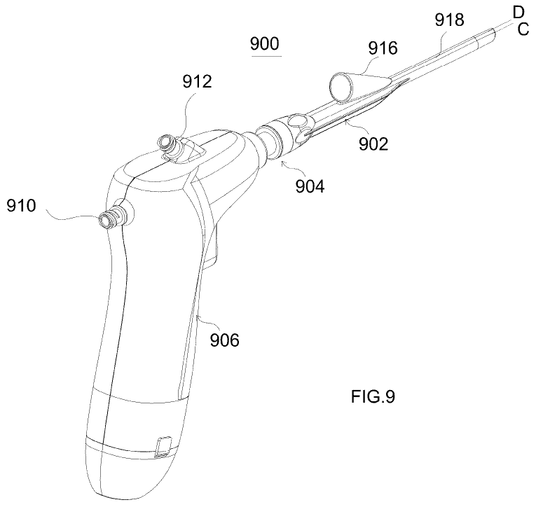

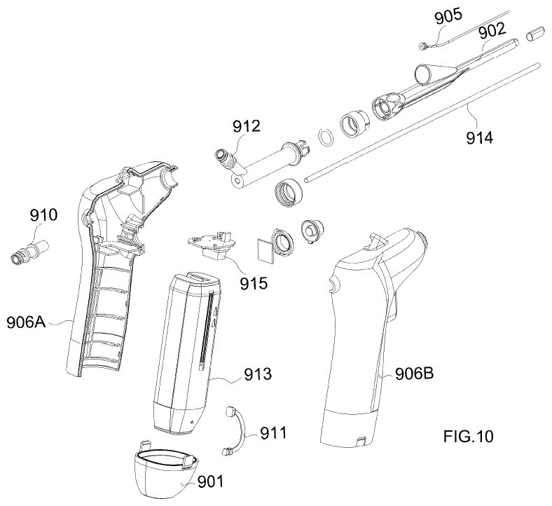

FIGS. 1-8 illustrate an example with an integrated display, and FIGS. 9 and 10 illustrate an example that uses a remote display.

FIG. 1 is a perspective view illustrating an example of a slim and ergonomic hand-held kit 100 comprising (i) an injection sheath 102 and (ii) an endoscope comprising a cannula 104, handle 106 and display 108 mounted to the handle. Preferably, at least one of cannula 104 and display 108 is coupled to handle 106 to be removable therefrom by hand, without tools, or both are so mounted to handle 106. Display unit 108 can be mounted in a holder secured to handle 106 to slide and an out of the holder and to make electrical contact with circuitry inside the handle through mating electrical contact in the display and the holder (not shown).

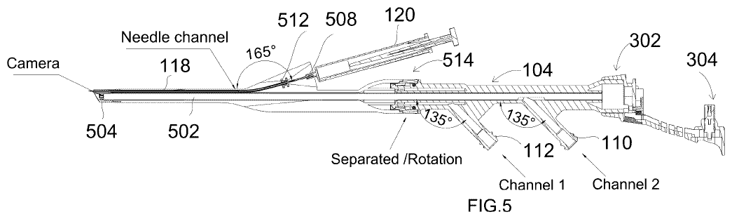

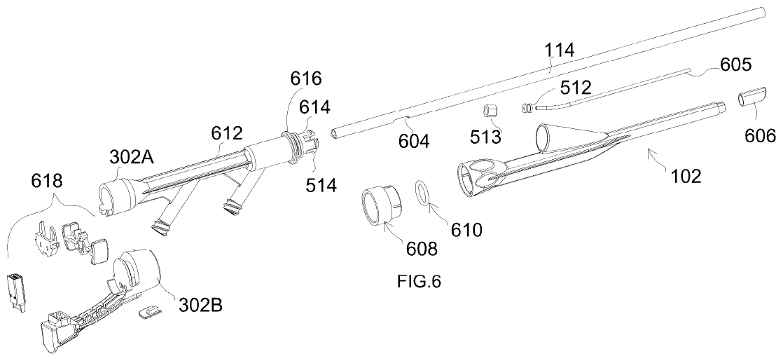

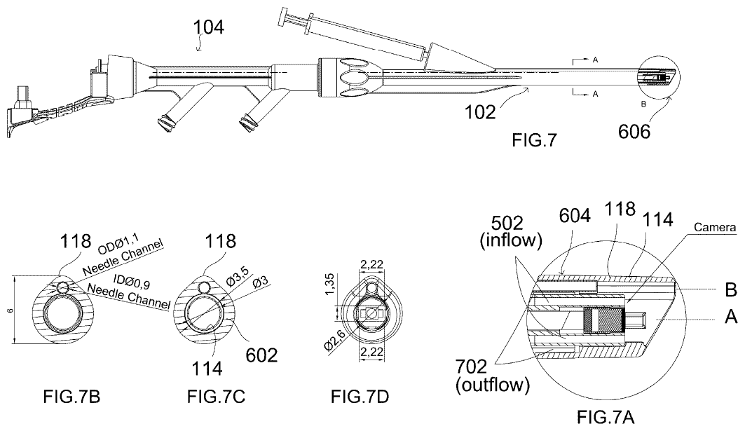

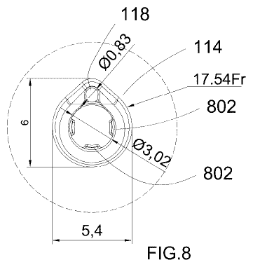

Injection sheath 102 comprises (i) an axially extending sheath tube 114 that has an internal channel, is open at its proximal and distal ends, and has a central axis A, (ii) an injection needle channel 118 that is open at its proximal and distal ends and has a central axis parallel to but radially spaced from axis A, and (iii) a needle entry port 116 that points up from sheath 102 and is coupled to needle channel 118 such that a needle 508 (FIG. 5) attached to a syringe 120 can pass through needle channel 118 and selectively exit distally from sheath 102 to inject an agent in a patient's internal passageway. The distal portion of sheath 102 comprises a tubular cap 606 (FIG. 6) that is fitted at the distal end of the sheath and has (i) a plurality of orientation landmarks 802 (FIG. 8) shaped as protrusions that extend inwardly and are circumferentially and radially spaced from each other, and (ii) a stopper 704 (FIG. 7A) that is proximal to orientation landmarks 802 and keeps cannula 104 spaced from target tissue to enable visualizing the target tissue, needle 508, and orientation landmarks 802.

Notably, sheath tube 114, needle channel 118, and needle entry port 116 are preferably integrally molded as a single unit of a biologically compatible plastic material.

An endoscope unit coupled with sheath 102 comprises (i) tubular cannula 104 with an internal channel 502 (FIG. 5) open at its distal end, (ii) an imaging module 504 (FIG. 5) at the cannula's distal end, and (iii) a fluid inflow port 110 coupled with internal channel 502 in the cannula and a fluid outflow port 112 coupled with a tubular space 702 (FIG. 7a) between cannula 104 and sheath 102. Fluid ports 110 and 112 extend down from a proximal portion of the cannula.

In kit 100, sheath 102 rotatably fits over a distal portion of cannula 104 such that (i) cannula 104 is proximal to stopper 704, (ii) imaging module 504 is proximal to and in position to image orientation landmarks 802 and a field distal from sheath 102 and preferably to also image a portion of needle 508 that may extend distally from sheath 102; (iii) the proximal portion of cannula 104, with inflow and outflow ports 110 and 112, is proximal to sheath 102; and (vi) cannula 104 and sheath tube 114 are dimensioned to provide a tubular space 702 between the cannula and the sheath for fluid flow therebetween that is coupled to outflow port 112 to aspirate fluid out of the patient's internal passageway.

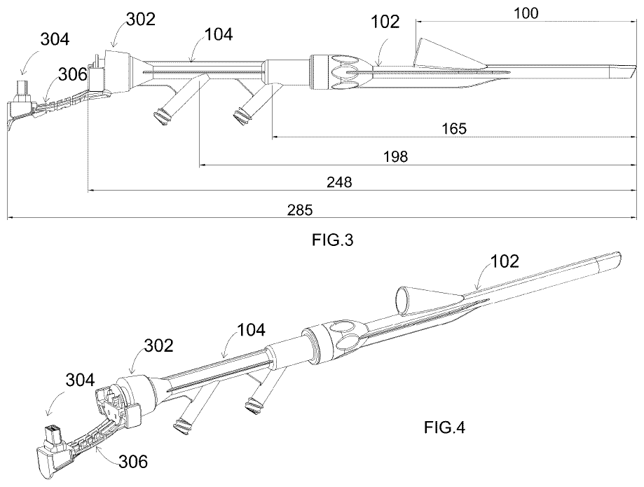

Handle 106 can releasably couple to cannula 104, using a mechanism that can be as described in detail in said U.S. Pat. No. 10,278,563 in connection with FIGS. 3 and 5A therein for coupling cannula 120 to handle 140. The coupling can be without allowing rotation of cannula 104 relative to handle 106, or it can enable rotation of cannula 106 relative to handle 106 about axis A, as in said U.S. Pat. No. 10,278,563. Handle 106 encloses on all sides the entirety of a processing electronics unit that is coupled with display 108 and imaging module 504 to receive image data from module 504 and process the data into images sent to display 108. The hand includes a removable cap that can be opened to withdraw the processing unit from the handle. An example of such handle and processing electronics unit is described in detail further below, in connection with FIG. 10.

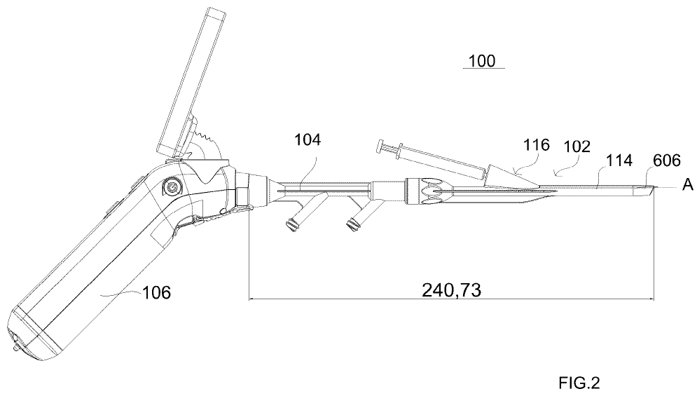

FIG. 3 is a side view and FIG. 4 is a perspective view of a cannula and an injection sheath of the system of FIGS. 1 and 2. The proximal portion of cannula 104 comprises a mechanical coupler 302 and an electrical coupler 304 connected via flexible cable 306 to the mechanical coupler. Electrical coupler 304 connects electrically to imaging module 504 through internal wiring (not shown) that can pass through internal channel 502 in cannula 104. Couplers 302 and 304 releasably connect cannula 104 to handle 106 as described further below and also in said U.S. Pat. No. 10,278,563. FIG. 3 shows dimensions that can be suitable for a specific example of the cannula and the sheath, but different dimensions can be selected depending on the intended use of the system 100 or preferences of users.

FIG. 5 is an axial sectional view of cannula 104 and sheath 102, according to some embodiments. Cannula 104 comprises an internal channel 502 that is open at its distal end and imaging module 504 at the distal end of channel 502. Needle 508 passes through needle port 116 and needle channel 118 and can protrude distally from the distal end of the needle channel. A backflow valve 512 keeps fluid from flowing back into needle port 116. A rotation mechanism 514 enables relative rotation of cannula 104 and sheath 102 about axis A. The rotation mechanism includes a ring seal to keep fluid from flowing proximally out of the mechanism. The external diameter of the cannula portion that is in sheath 104 and the internal diameter of the sheath are dimensioned to leave a tubular space 702 (FIG. 7A) between them. Fluid can flow proximally into tubular space 702 through the open distal end of sheath 102, through tubular space 702, and out of outflow port 112. Fluid can enter inflow port 110, flow distally through internal channel 502, and exit distally through the distal end of cannula 104, around imaging module 504.

FIG. 6 is an exploded perspective view of cannula 104 and sheath 102, according to some embodiments. Cannula tube 114 has an opening 604 through which fluid can enter the tube from inflow port 110 when tube 114 is in place in the proximal portion of cannula 104. Coupling mechanism 514 includes prongs 614 at the distal end of the proximal portion of cannula 104 that releasably snap into a ferrule 608 that fits into the proximal end of sheath 102, with a washer 610 therebetween. A ring seal 616 helps against backflow at coupling mechanism 514. A tube 604 that preferably is made of a metal such as medical grade stainless steel can be used to line needle channel 118 used together with backflow seal 512 and a ferrule 513. The proximal portion of cannula 104 has radially projecting ribs 612, for example four such ribs that are circumferentially spaced from each other, to stiffen that portion of the cannula. A proximal portion of sheath 102 can have similar ribs 617. Mechanical coupler 302 can comprise a portion 302A that fits in portion 302B, and additional elements 618 for coupling.

As noted above, tube 118, needle channel 118, and needle port 120 of sheath 102 are molded as a single, integral unit. Metal tube 605, which lines needle channel 118 is molded in place in needle channel 118 as the entire integral unit is being molded. For that purpose, metal tube 605 is positioned in a mold cavity, the mold is closed, and then the mold material is injected into the cavity to form channel 118 around the metal tube. As a result, metal tube 605 is held firm in needle channel 118, in firm contact with the needle channel. Metal tube 605 thus provides a smooth path for needle 508, and the sharp distal end of needle 508 avoids snagging plastic material in the needle channel.

FIG. 7 is a partial side view of cannula 104 and sheath 102 and partial axial section of cap 606, FIG. 7A is an enlarged axial sectional view of cap 606, FIGS. 7B-D are sectional views, and FIG. 8 is a view from the distal toward the proximal end of sheath 102 with cannula 104 therein and shows orientation landmarks 802, according to some embodiments. Cap 606 forms the distal end of sheath 102 and has a stopper 704 that keeps cannula tube 104 and imaging module 502 spaced proximally from the distal end of sheath 102. Camera module 502 thus is proximal to orientation landmarks 802 seen in FIG. 8. Imaging module 502 therefore views one or more of orientation landmarks 802 in addition to viewing tissue distal from sheath 102 and, if desired, a portion of needle 508 that protrudes enough distally from needle channel 118. Orientation landmarks 802 can be shaped to differ from each other so that images taken with imaging module 502 can easily tell a user how sheath 102 is angled around axis A relative to cannula 104.

FIG. 7A shows tubular space 702 between the outside of cannula tube 114 and the inside of sheath 102. Aspiration fluid can flow in this tubular space 702. FIG. 7A also shows central axis A of cannula 104 and central axis B of needle channel 118, which axis B is parallel to and radially spaced from axis A.

FIGS. 7B-D show cross-sections through the portion of cannula 104 that is inside sheath 102, with dimensions that are an example although other dimensions can be used. In the illustrated example, the inside diameter of the channel in sheath 102 is 3.5 mm and the outside diameter of cannula 104 is 3 mm, to thereby form tubular space 702. The examples of dimensions in FIGS. 7B-D are in mm, as are all other dimensions in other drawings, but the kit can use other dimensions depending on intended use and user preferences.

FIG. 8 shows an example of orientation landmarks 808 and shows dimensions as an example although other dimensions can be used. In the illustrated example, the cross-sectional external dimensions of the distal portion of sheath 102 that is intended to enter a patient's passageway are 6 mm at the diameter that includes needle channel 118 and 5.4 mm elsewhere.

FIGS. 9 and 10 illustrate an example of a kit that uses an outside display.

FIG. 9 is a perspective view of a kit 900 that comprises a sheath 902 that can be like sheath 102 in all respects and a cannula 904 that extends in a straight line along axis C from an entry port 910 at the proximal end of handle 906 toward the distal end of sheath 902. Port 910 can be an inflow port that leads to an inside channel in cannula 904. Port 910 and the cannula's internal channel also or alternatively can be used to pass surgical instruments such as a grasper or other instruments. An outflow port 916 extends up from handle 906 and leads to a tubular space between cannula 904 and sheath 902 that is like tubular space 702.

FIG. 10 is an exploded view of components of kit 900. For conciseness, elements that are like those of kit 100 are not numbered. As seen in FIG. 10, the handle comprises half shells 906A and 906B that are affixed to each other to form the grip of handle 906 that is like handle 106 in FIG. 1 in other respects. The bottom portion of this grip comprises a bottom cap 901 that clips and unclips to the open bottom of the grip and can be secured in place with a tie 911. Processor electronics 913 are in the form of an elongated module that slips up through the open bottom of handle 906 after bottom or end cap 901 is unclipped. Bottom or end cap 901 thereafter clips on so that handle encloses processor electronics 913 in their entirety and is sealed to keep fluids and other contaminants from reaching processor electronics 913. Handle 106 (FIG. 1) can be the same as handle 906 in these respects. Processing electronics unit 913 further includes a wireless transmitter/receiver for communicating wirelessly with an external display (not shown) that can serve the function of display 108 (FIG. 1) but can be physically separated from handle 906. FIG. 10 further shows an electrical connector 915 secured to handle 906. Processor unit 913 included in its upper portion a matching electrical connector (not shown) that mates with connector 915 when processing unit 913 slide up to a resting position in handle 906.

An example of injecting medication in a patient's internal passageway under endoscopic guidance according to the disclosure above comprises assembling the kit in the form shown in FIG. 1 or 9, if not already so assembled. The user introduces a distal portion of injector sheath 102 or 902, with endoscope in place in the sheath, into a patient's passageway while observing surrounding tissue on display 108 and/or on a remote display and concurrently observing orientation landmarks 802 and thus the relative angular orientation of the sheath and cannula and preferably also observing a distal portion of the injection needle that may protrude distally from the sheath. When the distal end of the sheath is at a desired position relative to target tissue and an injection site, the user pushes the injection needle with the syringe to penetrate tissue such as at the urethra wall along a path generally parallel or nearly parallel to the urethra wall at or near the injection site. Because the injection sheath distal end typically is wider than the urethra, tissue just distal to its distal end tends to bulge radially inwardly, in the natural path of the injection needle, so that moving the needle distally tends to make it enter path in tissue that is nearly parallel to the urethra wall at the bulge. If there is no bulge or an insufficient bulge, the user can tilt the sheath by tilting the handle relative to the patient to select an appropriate path for the injection needle. After an injection, the user can rotated the sheath relative to the cannula around the cannula's central axis and/or can rotate the handle around the same axis and/or move the handle distally or proximally relative to the patient to thereby position the needle for an injection at another site. Alternatively, the kit can be withdrawn from the patient after a single injection. After the kit is out of the patient, a user can open the bottom cap and take the processing unit from the handle, for use in another procedure after cleaning. If the kit includes a display mounted to the handle, the user slides the display out of the handle for use in another procedure. In each case, the user disposes of the remainder of the kit as medical waste.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered as illustrative and not restrictive, and the body of work described herein is not to be limited to the details given herein, which may be modified within the scope and equivalents of the appended claims.

1. A kit for a medical procedure that includes injecting an agent at one or more injection sites in a patient's internal passageway, comprising:
an injection sheath that includes (i) a sheath tube with open distal and proximal ends and a central axis, (ii) an injection needle channel with a central axis parallel to but radially spaced from the tube's central axis, and (iii) a needle entry port that is at a proximal portion of the sheath and is coupled to the needle channel;
wherein the sheath tube, the needle channel, and the needle entry port are integrally molded as a single unit of a biocompatible plastic material;
a tubular cap that is fitted to the distal end of the sheath tube and has (i) a plurality of orientation landmarks that extend radially inwardly and are circumferentially and radially spaced from each other and (ii) a stopper that is axially proximal to the orientation landmarks;
an endoscope unit that includes (i) a tubular cannula with an internal channel open at a distal end of the cannula, (ii) an imaging module at the cannula's distal end, (iii) fluid inflow and fluid outflow ports at a proximal portion of the sheath, with the inflow port coupled to the cannula's internal channel;
wherein the sheath tube rotatably fits over a distal portion of the cannula;
wherein (i) the cannula is proximal to the stopper, (ii) the imaging module is proximal to and in a position to image the orientation landmarks and a field distal from the sheath, (iii) the proximal portion of the cannula, with the inflow and outflow ports, is proximal to the sheath, and (iv) the cannula and the sheath tube are dimensioned to provide a tubular space for fluid flow therebetween that is coupled to the outflow port and has an open distal end.

2. The kit of claim 1, further including (i) a handle to which the proximal portion of the cannula is releasably coupled mechanically and electrically, the handle comprising a hollow pistol grip extending in a downward direction and shaped and dimensioned to be grasped by a user's hand and having a removable end cap closing off a bottom portion of the grip, (ii) a display mounted to the handle, and (iii) processor electronics housed in their entirety in the hollow grip and electrically coupled with the imaging module and the display to receive image data from the imaging module and to display images based thereon on the display.

3. The kit of claim 2, in which the handle end cap is removable without tools to enable the processing electronics to slide up into the handle grip and the cap is configured to re-attach to the grip so the grip encloses the entirety of the processing electronics.

4. The kit of claim 1, in which the sheath further comprises a tube made of a medical grade metal that lines the needle channel to facilitate distal motion therein of an injection needle with a sharp distal end, wherein the metal tube is molded into the needle channel while molding the sheath tube, the needle port, and the sheath tube in the same mold.

5. The kit of claim 1, in which the cannula's proximal portion has a plurality of axially elongated stiffening ribs that extend radially relative to the central axis and are circumferentially spaced from each other.

6. The kit of claim 5, in which the sheath's proximal portion has a plurality of axially elongated stiffening ribs that extend radially relative to the central axis and are circumferentially spaced from each other.

7. The kit of claim 1, in which the sheath's proximal portion has a plurality of axially elongated stiffening ribs that extend radially relative to the central axis and are circumferentially spaced from each other.

8. The kit of claim 1, in which the external diameter of the cannula's distal portion and the internal diameter of the sheath differ by 0.5 mm to thereby form the tubular space for fluid flow therein.

9. The kit of claim 1, in which the sheath has a diameter that is 6 mm when intersecting the needle channel and 5.4 mm elsewhere.

10. A method comprising:
introducing into a patient's passageway an injection sheath with open distal and proximal ends and an injection needle channel with a central axis parallel to but radially spaced from a central axis of the sheath and a needle entry port at a proximal portion of the sheath coupled to the needle channel;
including in a distal portion of the sheath a tubular cap that has a plurality of orientation landmarks extending radially inwardly and circumferentially and radially spaced from each other and a stopper that is axially proximal to the orientation landmarks;
providing an endoscope inserted in the sheath and having an internal channel open at a distal end, an imaging module at the endoscope's distal end, and fluid inflow and fluid outflow ports at a proximal portion of the endoscope, with the inflow port coupled to the endoscope's internal channel;
selectively rotating by hand the sheath and the endoscope relative to each other while the endoscope is proximal to the stopper, the imaging module is proximal to and in a position to image the orientation landmarks and a field distal from the sheath, the proximal portion of the endoscope, with the inflow and outflow ports, is proximal to the sheath;
wherein the endoscope and the sheath are dimensioned to provide a tubular space for fluid flow therebetween coupled to the outflow port and having an open distal end; and
passing an injection needle through the needle channel such that a sharpened distal end of the needle protrudes distally from the sheath and injecting an agent into tissue through the needle.

11. The method of claim 10, including lining the needle channel with a tube made of surgical grade metal to facilitate distal motion therein of the sharpened distal end of the needle.

12. The method of claim 10, including viewing images based on image data from the image module on a display mounted to a handle to which the endoscope is releasably coupled.

13. The method of claim 12, including inserting processing electronics into the handle after removing without tools a bottom cap of the handle, closing the bottom cap to enclose the entirety of the processing electronics in the handle, carrying out a medical procedure with the kit, opening the bottom cap and removing the processing electronics from the kit, removing the display without tools, and disposing of the remainder of the kit as medical waste.

14. A kit for a medical procedure that includes injecting an agent at one or more injection sites in a patient's internal passageway, comprising:
an injection sheath that comprises a sheath tune with open distal and proximal ends and a central axis, an injection needle channel with a central axis parallel to but radially spaced from the sheath's central axis, and a needle entry port that is at a proximal portion of the sheath and is coupled to the needle channel;
wherein the sheath tube, the needle channel, and the needle entry port are integrally molded as a single unit of a biocompatible plastic material;
wherein the sheath includes a tubular cap that is fitted to the distal end of the sheath tube and has a plurality of orientation landmarks that extend radially inwardly and are circumferentially and radially spaced from each other and a stopper that is axially proximal to the orientation landmarks;
an endoscope unit that includes a tubular cannula with an internal channel open at a distal end of the cannula and an imaging module at the cannula's distal end;
wherein the sheath and the cannula are configured to rotate by had relative to each other about the cannula's central axis and the sheath tube has an inside diameter greater than the cannula's outside diameter to thereby form a tubular space therebetween for passage of fluid; and
a handle with an inflow port at a proximal end of the handle coupled to the cannula's internal channel and an outflow port extending above the handle and coupled to said tubular space;
wherein the cannula is proximal to the stopper and the imaging module is proximal to and in a position to image the orientation landmarks and a field distal from the sheath.

15. The kit of claim 14, in which the handle comprises a hollow pistol grip extending in a downward direction and shaped and dimensioned to be grasped by a user's hand and having a removable end cap closing off a bottom portion of the grip, and processor electronics housed in their entirety in the hollow grip and electrically coupled with the imaging module and the display to receive image data from the imaging module and to display images based thereon on the display.

16. The kit of claim 14, in which the sheath further comprises a tube made of a medical grade metal that lines the needle channel to facilitate distal motion therein of an injection needle with a sharp distal end, wherein the metal tube is molded into the needle channel while molding the sheath tube, the needle port, and the sheath tube in the same mold.

17. The kit of claim 14, in which the cannula's proximal portion has a plurality of axially elongated stiffening ribs that extend radially relative to the central axis and are circumferentially spaced from each other.

18. The kit of claim 17, in which the sheath's proximal portion has a plurality of axially elongated stiffening ribs that extend radially relative to the central axis and are circumferentially spaced from each other.

19. The kit of claim 14, in which the sheath's proximal portion has a plurality of axially elongated stiffening ribs that extend radially relative to the central axis and are circumferentially spaced from each other.

20. The kit of claim 14, in which the external diameter of the cannula's distal portion and the internal diameter of the sheath differ by 0.5 mm to thereby form the tubular space for fluid flow therein.
